(19) United States
(12) Patent Application Publication
(10) Pub. No.: US 2023/0338545 A1
(43) Pub. Date: Oct. 26, 2023

(54) COMPLEXES OF MAGNESIUM MALTOL (3-HYDROXY-2-METHYL-4H-PYRAN-4-ONE) FOR ORAL SUPPLEMENTATION

(71) Applicants: Robert P. Doyle, Manlius, NY (US); Ren A. Gonzalez, Nibley, UT (US); Derek R. Case, Syracuse, NY (US)

(72) Inventors: Robert P. Doyle, Manlius, NY (US); Ren A. Gonzalez, Nibley, UT (US); Derek R. Case, Syracuse, NY (US)

(73) Assignee: SYRACUSE UNIVERSITY, Syracuse, NY (US)

(21) Appl. No.: 18/026,657

(22) PCT Filed: Sep. 17, 2021
(86) PCT No.: PCT/US2021/050796

§ 371 Date: Mar. 16, 2023

Related U.S. Application Data
(60) Provisional application No. 63/079,743, filed on Sep. 17, 2020.

(51) Int. Cl. A61K 47/54; A61K 9/00; A61K 47/12

(52) U.S. Cl. CPC A61K 47/545; A61K 9/0053; A61K 47/12

(57) ABSTRACT

A complex of magnesium maltol that can deliver magnesium via oral administration at a low cost. The magnesium maltol is formed by dissolving an amount of maltol in water. A solution of citric acid and magnesium oxide is added to the maltol, allowed to react, and then dried to obtain magnesium maltol. The approach may also be used to produce magnesium ethylmaltol. Cellular uptake studies demonstrate that both magnesium maltol and magnesium ethylmaltol provide a substantial uptake of magnesium and thus the complexes offer a route for magnesium supplementation such as that needed by patients with hypomagnesemia.

### BACKGROUND OF THE INVENTION

### 1. Field of the Invention

The present invention relates to magnesium supplements and, more specifically, to a complex of magnesium and maltol for oral treatment of magnesium deficiency.

### 2. Description of the Related Art

Magnesium deficiency, known as hypomagnesemia, occurs when the amount of magnesium in the blood is lower than normal. Magnesium is an essential mineral and a cofactor for hundreds of enzymes, and is involved in numerous pathways including energy production, nucleic acid and protein synthesis, ion transport, and cell signaling. As a result, inadequate dietary intakes or low serum concentrations of magnesium has been associated with increased risk of cardiovascular disease, osteoporosis, and various metabolic disorders. The conventional approach for the treatment of hypomagnesemia is to administer magnesium orally or intravenously. However, current oral supplements are not as useful as potential magnesium chelates. Thus, there is a need in the art for a magnesium chelate complex that can be delivered orally at a low cost.

### BRIEF SUMMARY OF THE INVENTION

The present invention comprises complexes of magnesium maltol that can deliver magnesium via oral administration at a low cost. The magnesium maltol is formed by dissolving an amount of maltol in water. A solution of citric acid and magnesium oxide is added to the maltol, allowed to react, and then evaporated to obtain magnesium maltol.

### BRIEF DESCRIPTION OF THE SEVERAL VIEWS OF THE DRAWING(S)

The present invention will be more fully understood and appreciated by reading the following Detailed Description in conjunction with the accompanying drawings, in which:

FIG. 1 is a diagram of the structure of magnesium maltol according to the present invention;

FIG. 2 is a diagram of a method for synthesizing magnesium maltol according to the present invention;

FIG. 3 is a diagram of a method for synthesizing magnesium ethylmaltol according to the present invention;

FIG. 4 are graphs comparing the FT-IR spectra of maltol and Complex 1 (Left) and ethylmaltol and Complex 2 (Right), where the insets at right are a zoomed display of the region between 500-1700 cm⁻¹ to emphasized changes observed in the region attributed to the ketone fingerprint (1500-1700 cm⁻¹); and

FIG. 5 is a graph of cellular uptake of MgCl₂, Complex 1, and Complex 2 in CaCo-2 cells.

### DETAILED DESCRIPTION OF THE INVENTION

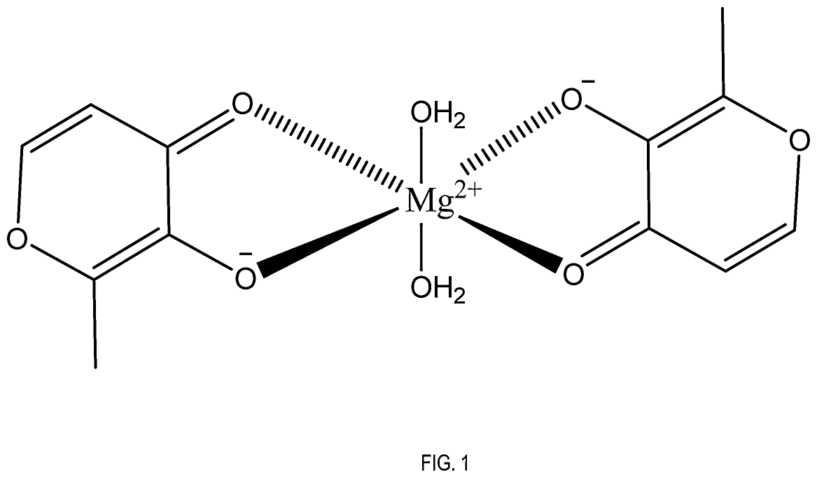

Referring to the figures, wherein like numeral refer to like parts throughout, there is seen in FIG. 1 a diagram of the structure and composition of magnesium maltol. Magnesium maltol synthesis was conducted from MgO and maltol. Both proton and carbon NMR indicate that maltol coordinates to magnesium. The disappearance of C₅, and significant reduction of C₁, carbon signals confirm coordination through carbonyl and alcohol moieties. ESI-MS confirms 1:2 (Mg:Malt) stoichiometry. The solubility of magnesium maltol was 33.3 mg/mL at RT (maltol is 10.9 mg/mL at 15° C.), thus magnesium maltol is approximately three times more soluble in water than pure maltol.

### EXAMPLE 1

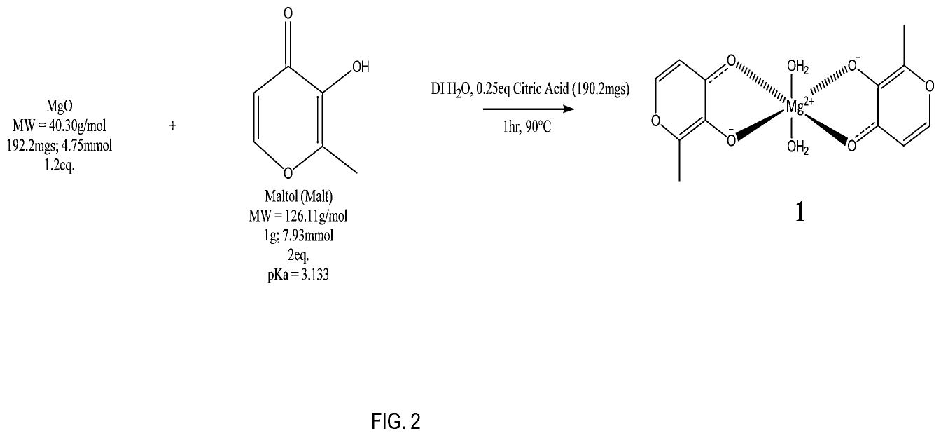

Referring to FIG. 2, magnesium maltol was synthesized as follows. A 1.0022 g sample of maltol (Malt—7.93 mmol; 2 eq.) was dissolved in approximately 10 mLs 18MΩ H₂O in a 50 mL round-bottom flask, with constant heating and stirring at 90° C. A separate solution of 160.5 mgs magnesium oxide (MgO—3.96 mmol; 1 eq.) was taken up in approximately 10 mLs 18MΩ H₂O, with an addition of 193.1 mgs citric acid (0.25 eq), constantly stirred and heated to 90° C. The MgO/CA solution was added to the maltol solution—upon addition, the combined solution turned a milky, white color (after 5 minutes, the solution was wholly soluble). The reaction was left to run for 1 hr at 90° C. The reaction was cooled to room temperature and filtered through a Buchner funnel (no solid was observed on the filter paper). The pH of the solution was found to be 9.85. The solution was concentrated via rotary evaporation—solid began to precipitate during this process. The solution was taken to dryness on the rotary evaporator and further dried overnight in vacuo. Yield was found to be approximately 50%. Proton NMR of the solution confirmed presence of magnesium maltol chelate when compared to resultant product of the synthetic approach utilizing magnesium oxide. Mass spectrographic analysis confirmed that the yielded product was likely due to the expected mass for [Mg(Malt)₂]¹⁺.CH₃OH as the mass spectrograph was conducted in CH₃OH. Nuclear magnetic resonance imaging taken in 1:6 (v:v) D₂O:H₂O demonstrates the formation of magnesium maltol signal. A slight upfield shift of all proton signals was expected given the electropositive shielding effect of the divalent magnesium center and predicted coordination to the conjugated system. As expected, an upfield shift of all maltol protons was observed, i.e., H₁=7.952 ppm (Δppm=0.03 ppm), H₂=6.474 ppm (Δppm=0.02 ppm) and H₃=2.331 ppm (Δppm=0.03 ppm). The residual peak integration observed for magnesium maltol was 5, which matched the expected integration signal of 5 (1:1:3). The residual peak integration suggests a 9.2-15.7% impurity, which could be free citrate or magnesium citrate. As explained below, the impurity is likely magnesium citrate. An overlay of the ¹H NMR of maltol and magnesium maltol taken in 1:6 (v:v) D₂O:H₂O shows an upfield shift of all proton signals, which is indicative of chelation. Proton signals between 2.5-2.7 ppm may be either citric acid or magnesium citrate.

A comparison of the ¹H NMR of citric acid, magnesium citrate, maltol and magnesium maltol suggests that proton signals of MgMalt between 2.5-2.7 ppm are the result of citric acid and that the MgMalt contains 9.2-15.7% magnesium citrate. Similarly, characterization via ¹³C NMR taken in 1:6 (v:v) D₂:H₂O, as seen in FIG. 7, shows significant reduction of C₁ signal and disappearance of C₅ signal, which is further support that magnesium has coordinated. The dashed blue lines show overlap of magnesium citrate carbon signals, and the carbon NMR of magnesium maltol, coupled with proton NMR of magnesium maltol, confirms the impurity is magnesium citrate rather than citric acid. A characterization of magnesium maltol via HSQC NMR taken in 1:6 (v:v) D₂O:H₂O revealed an assignment of H₃ that shows coupling to 14.25, indicating that this remains C₆ even when complexed. The observation of conservation of C₆ is expected given predicted coordination between the carbonyl and alcohol moieties. The observed secondary couplings are due to magnesium citrate. A characterization of magnesium maltol via HMBC NMR revealed an assignment of H₁ that shows coupling to 154.24 and 177.57, indicating that these are C₃ and C₁ respectively. The assignment of H₂ shows coupling to 111.74, 154.16 and 177.57, indicating that these are C₂, C₄, and C₁ respectively. H₃ shows coupling to 154.16 confirming assignment of C₄.

### EXAMPLE 2

The synthesis of magnesium maltol was further optimized to reduce the amount of unrelated maltol as seen in FIG. 2. A 1.00 g sample of maltol (7.93 mmol; 2 eq.) was dissolved in 10 mLs of DI H₂O in a 50 mL round-bottom flask, with constant stirring at 90° C. A separate solution of 192.2 mgs of magnesium oxide (MgO—mmol; 1.2 eq.) was taken up in 10 mLs of H₂O, with an addition of 190.5 mgs of citric acid (CA—0.25 eq), constantly stirred and heated to 90° C. The MgO/CA solution was subsequently added to the maltol solution in small increments over ˜5 min. Upon addition, the mixture was a translucent white color that solubilized in about 30 seconds; each subsequent addition was administered when the previous addition had become wholly soluble. After all additions, the reaction was noted as colorless and clear. The reaction was conducted for one hour, whereupon the solution was noted as yellow and clear. The reaction was allowed to cool to room temperature and the pH was noted as 9.85. The solution was dried in vacuo—producing a tan solid, which was used for subsequent analyses. The yield of Complex 1 was stoichiometric relative to maltol with a purity of 91.2% based on ¹H NMR. The solubility of Complex 1 was determined to be 3.33 mg/100 mL H₂O. Drying could occur in any number of conventional ways, including spray drying.

### EXAMPLE 3

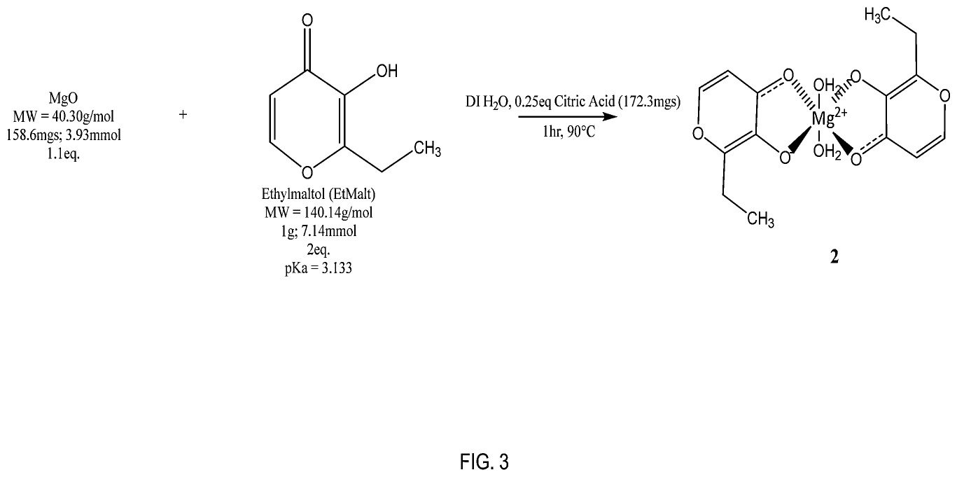

The synthesis approach of magnesium maltol was additionally used to form magnesium ethylmaltol as seen in FIG. 3. A 1.01 g sample of ethylmaltol (EtMa—7.14 mmol; 2 eq.) was dissolved in 10 mLs of DI H₂O in a 50 mL round-bottom flask, with constant stirring at 90° C. A separate solution of 158.6 mgs magnesium oxide (MgO—3.93 mmol; 1.1 eq.) was taken up in 10 mLs of DI H₂O, with an addition of 172.3 mgs of citric acid (CA—0.25 eq), constantly stirred and heated to 90° C. The MgO/CA solution was subsequently added to the ethylmaltol solution in small increments over 5 min. Upon addition, the mixture was a translucent white color that solubilized in about 30 seconds; each subsequent addition was administered when the previous addition had become wholly soluble. After all additions, the reaction was noted as colorless and clear. The reaction was conducted for one hour, whereupon the solution was noted as clear and amber/orange in color. The solution was allowed to cool to room temperature and the pH was noted as 9.85. The solution was dried in vacuo, at which time a tan solid was observed. The yield was found to be stoichiometric relative to ethylmaltol, and the purity was 92.1% based on ¹H NMR. The solubility of Complex 2 was determined to be 26.8 g/100 mL H₂O.

### Results

Both Complex 1 and 2 were synthesized from a magnesium oxide starting material in the presence of citric acid to aid in the solubility of the relatively water-insoluble metal oxide; the citric acid provides a proton. Addition of citric acid at 0.25 equivalents was the lowest concentration found that could drive the reaction while also minimizing the formation of magnesium citrate, with 1H NMR of both Complex 1 and 2 indicating ˜8% magnesium citrate in the final products. Increasing the equivalents of magnesium oxide to 1.2 eq. and 1.1 eq for the synthesis of Complex 1 and 2, respectively, was required to mitigate the return of unreacted maltol or ethylmaltol. Specifically, at 1:2 equivalents of magnesium oxide:maltol, upon cooling the solution from reaction temperature, a white precipitate was observed. Analysis of the dried precipitate via EA confirmed it to be unreacted maltol. Given the requirement to have citric acid present to drive the reaction, minimized as it is to 0.25 eq, it is clear the citrate is outcompeting maltol for magnesium binding. Thus, an additional stoichiometric amount of MgO is necessary to drive complete chelation of all maltol starting material.

The same requirement for an excess of MgO is observed for the synthesis of Complex 2. The only difference noted was the requirement to cool the reaction to −20° C. to recover the unreacted ethylmaltol, a requirement given the increased water solubility of ethylmaltol (5.84 g/100 mL) relative to maltol (1.2 g/100 mL). The presence of unreacted ethylmaltol was confirmed via EA.

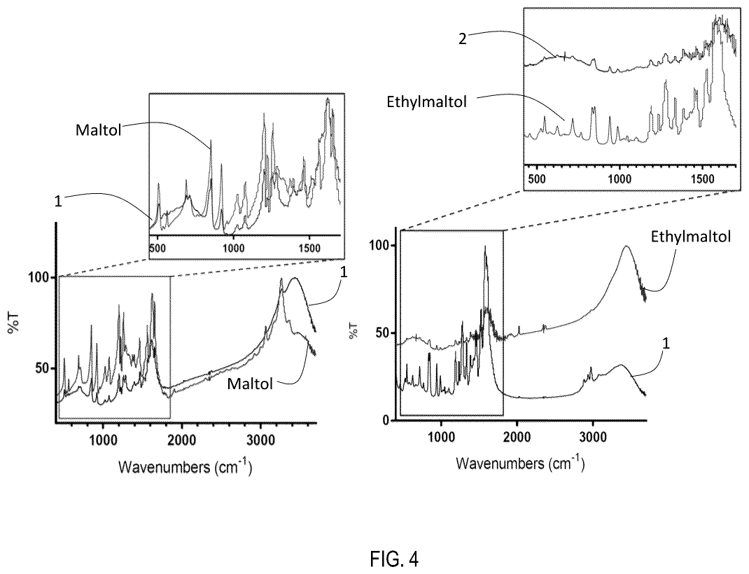

The infrared spectra of Complex 1 and 2 were compared to the infrared spectra of both maltol and ethylmaltol, as seen in FIG. 4. FT-IR of both ligands showed changes to frequency regions that corresponded specifically to the —OH stretching mode of both ligands associated with coordination of this moiety. There is a slight change observed in the frequency of the signals attributed to the ketone moiety of Complex 1 to higher energy relative to maltol.

This suggests magnesium coordination about the ketone, and a shift to slightly higher energy is consistent with magnesium coordination as reported by others. However, this is different than the observed signal shifts observed for other divalent metal-maltol complexes such as bismaltolato zinc (II). Upon coordination to zinc, the infrared maltol signals attributed to the ketone moiety are shifted to lower energy. This may be the result of zinc being less electropositive in character than magnesium, thus resulting in less ionic character upon coordination, but may also be attributed to differences in ionic radii of the two metals. No significant change to the region associated with the ketone is observed for Complex 2. However, coordination about this site is again supported by ¹³C NMR. Additionally, the spectra of both Complex 1 and 2 indicate the presence of coordinated water signified by broad signals observed between 3200-3500 cm⁻¹, as were observed for the previously describe zinc maltol complexes. Further insight into the conclusions drawn from the FT-IR spectra are provided in Table 1

TABLE 1
Assignment of the infrared spectra values of maltol, Complex
1, ethylmaltol, and Complex 2 - additional shifting upon
ligand coordination to magnesium is also provided

| | IR Frequency | | Change |
|---|---|---|---|
| Complex | (cm⁻¹) | Assignment | (cm⁻¹) |
| Maltol | 3260 | v(OH), C—OH | — |
| | 1655 | v(C═O) | — |
| | 1621 | v(C═O) | — |
| Complex 1 | 3435 | v(OH), H₂O | — |
| | 3264 | v(OH), C—OH | 4 |
| | 1655 | v(C═O) | — |
| | 1617 | v(C═O) | 4 |
| Ethylmaltol | 3369 | v(OH), C—OH | — |
| | 1617 | v(C═O) | — |
| | 1525 | v(C═O) | — |
| Complex 2 | 3447 | v(OH), H₂O | — |

Thermal analysis of Complex 1 was conducted relative to maltol. Maltol exhibited a continuous percent weight loss from onset at ˜70° C. to 200° C. and stopped decreasing in percent weight at approximately 5%, thus suggesting decomposition of maltol between 160 and 200° C., which is consistent with the known melting point of maltol at 160° C. TGA analysis of Complex 1 exhibited a similar decomposition trend differing only with the percent weight loss exhibited by Complex 1 reaching a minimum at approximately 40%. The DSC spectrum of Complex 1 exhibited two endotherms: a broad endotherm with an apex at approximately 120° C. attributed to the loss of coordinated water from Complex 1, and a secondary more intense, sharper endotherm attaining apogee at approximately 160° C. This endotherm is attributed to the thermal decomposition of the maltol ligand, which is consistent with the TGA of maltol. The endotherm at 120° C. corresponds to a percent weight decrease of 22.70% observed on the TGA of Complex 1, which is attributed to the loss of four water molecules given a predicted percent weight change of 20.80%. While the EA of Complex 1 suggests only three waters, this difference is attributed to different hydrated states given the propensity of magnesium to take on water.

As observed with maltol, ethylmaltol exhibited only one continuous percent weight decrease from approximately 70° C.-200° C. and stops decreasing in weight at approximately 5% weight. This profile is attributed to the thermal decomposition of the ethylmaltol ligand, which is predicted to be roughly the same as maltol at ˜160° C. The TGA of 2 differed to that of ethylmaltol in that it exhibited two distinguishable percent weight decreases and stopped decreasing in percent weight at approximately 35%. Both percent weight changes correspond to two separate endotherms observed on the DSC of Complex 2—one broad endotherm apexed at approximately 110° C. and a secondary sharp, and substantially more intense, endotherm with an apex at approximately 320° C., respectively. The first broad endotherm observed on the DSC of Complex 2 shows a corresponding percent weight change of 15.33%, which corresponds to the loss three waters from the overall [Mg(EtMa)₂(H₂O)z₂].H₂O] complex supported by the EA with a predicted weight percent change of 15.15%. The secondary, more intense, endotherm at approximately 320° C. is attributed to the decomposition of the ethylmaltol ligand. The number of waters observed for Complex 1 via thermal analyses predict two waters directly coordinated to the magnesium core, and two additional waters of crystallization. The presence of three waters is consistent with EA. However, magnesium readily absorbs water and differing drying conditions and/or sample preparations likely have contributed to the different hydration states noted. The three waters observed for 2 support two coordinated waters and one water of crystallization.

Mg²⁺ readily coordinates with hard Lewis bases as exemplified by the monodentate magnesium chelates of formic acid, orotic acid, maleic acid, the bidentate magnesium chelates of mandelic acid and malic acid, and the tridentate magnesium chelate of citric acid. Ligand chelation to the divalent magnesium cation is often characterized by an observable shift in the NMR, or a change in signal resolution, of the proton signals adjacent to the Lewis bases of the ligand, due to the electropositive character of the metal. Given the impact that concentration and pH may have on shifting of proton and carbon signals, each sample of Complex 1 and 2 was analyzed at equimolar concentrations and at identical pH to maltol and ethylmaltol, respectively, with the instrument internally calibrated to TMS and each spectrum calibrated to the residual HOD peaks present in the D₂O solvent.

¹H NMR was conducted on both maltol and Complex 1 in 700 μl of D₂O. At equimolar concentrations, the integration of maltol and Complex 1 is conserved. Additionally, Complex 1 showed a small but observable upfield shift for all three protons of 0.04 ppm for H₂, 0.01-0.02 ppm for H₃, and 0.03 ppm for H₃. There is substantial sharpening of all three proton signals for 1 relative to free maltol.

Both heteronuclear single quantum coherence (HSQC) and heteronuclear multiple bond correlation (HMBC) confirmed the proton and carbon signal assignments of maltol, showing that C₁ was the most downfield carbon signal at 175.20 ppm, while C₅ was assigned at 154.50 ppm, and C₂ was assigned at 113.40 ppm. Evaluation of maltol ¹³C NMR (comparatively to Complex 1 showed a significant reduction in intensity, as well as broadening of the C₁ and C₂ carbon signals. Analysis also showed a complete disappearance of the signal attributed to C₅; a similar trend was observed for the ¹³C NMR of Complex 2, except for C₅ peak intensity. Additionally, there was an observable downfield shift of the C₁ carbon signal (178.20 ppm) and an upfield shift of the C₂ (112.40 ppm) carbon signal.

¹H NMR was conducted on ethylmaltol and the pure and dried 2 in D₂O. At equimolar concentrations, the integration of ethylmaltol and Complex 2 is conserved. Additionally, Complex 2 showed a small observable upfield shift for each of the proton peaks of 0.02 ppm, 0.01 ppm, 0.01 ppm, and 0.02-0.03ppm for H₁-H₄, respectively, a trend similar to that noted for Complex 1. Assignments of all proton and carbon signals were confirmed via 2D ¹H-¹³C NMR.

Over triplicate independent runs, the solubility of 1 was found to be 3.33±0.19 g per 100 mL of H₂O, and the solubility of 2 was found to be 28.4±0.86 g per 100 mL of H₂O. The solubility of 1 is approximately 2.8× greater than that of maltol (1.2 g/100 mL) and the solubility of 2 is approximately 4.9× greater than that of the ethylmaltol ligand (5.84 g/100 mL) (Table 2). These solubilities are consistent with the reported solubilities of maltol and ethylmaltol.

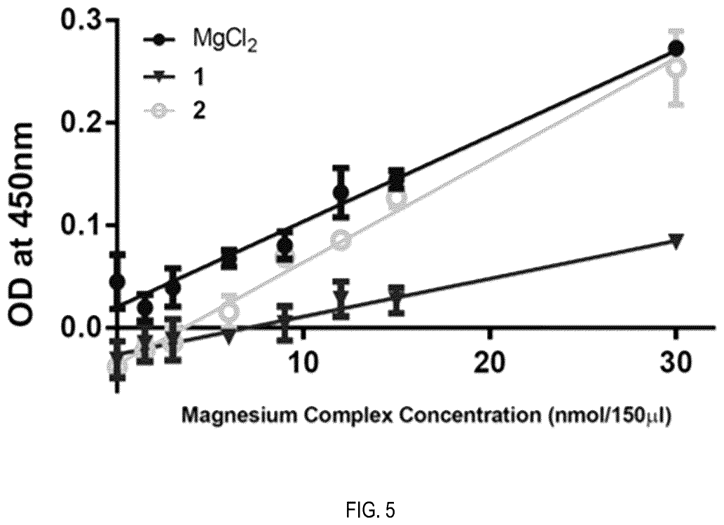

Cellular uptake of Complex 1 and 2 were conducted in CaCo-2 cells at an incubation time of 1 hr as seen in FIG. 5. Uptake was evaluated with the understanding that both Complex 1 and 2 contained ˜8% magnesium citrate, and the concentrations are based upon a total concentration of magnesium contributed from both species as based upon molecular weight. Both compounds provided substantial uptake of magnesium, with Complex 2 showing slightly greater uptake than Complex 1, at a lower percent magnesium composition (7.2% versus 7.8%, respectively).

Hypomagnesemia is a greatly under-appreciated clinical issue, and is common in critically ill patients, where it may lead to complications, from severe to fatal. Magnesium compounds that are fully characterized and which have the properties and benefits of being readily water soluble, all natural/GRAS and readily absorbed, is a current unmet need. Such compounds both offer ready incorporation into supplements, but also have scope to become magnesium pharmaceuticals, which can be used in a clinical setting to off-set side-effects of magnesium deficiency such as cardiovascular and neuromuscular manifestations. The present invention provides for the syntheses of magnesium maltol (Complex 1) and magnesium ethylmaltol (Complex 2). Solution state and solid-state characterization enabled full characterization of both complexes and analysis of cellular uptake data in the human CaCo-2 cell line confirmed cellular entry. Given the characterization, water solubility, cell uptake and all natural/GRAS status of the ligands (and magnesium oxide and citric acid starting materials), these compounds offer great opportunities as food/supplement ingredients and for potential future pharmaceutical development.

What is claimed is:

1. An oral supplement, comprising a coordinate of a magnesium and a maltol.

2. The oral supplement of claim 1, wherein the coordinate of the magnesium and the maltol has the structure

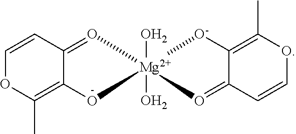

3. The oral supplement of claim 1, wherein the coordinate of the magnesium and the maltol has the structure

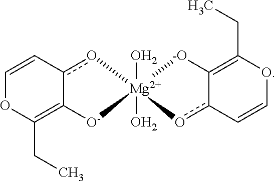

4. A method of making an oral supplement, comprising the steps of:
dissolving an amount of a maltol in water to form a first solution;
adding an amount of citric acid to an amount of magnesium oxide to form a second solution;
mixing the first solution and the second solution to form a combined solution; and
drying the combined solution to obtain a magnesium supplement.

5. The method of claim 4, wherein the magnesium supplement comprises magnesium maltol.

6. The method of claim 4, wherein the magnesium supplement comprises magnesium ethylmaltol.

7. The method of claim 4, wherein the step of dissolving the amount of the maltol in water to form a first solution comprises dissolving one gram of the maltol per 10 milliliters of water.

8. The method of claim 7, wherein the step of dissolving an amount of the maltol in water to form a first solution includes stirring at a temperature of 90 degrees Celsius.

9. The method of claim 4, wherein the step of adding an amount of citric acid to an amount of magnesium oxide to form a second solution comprises adding 192.2 milligrams of magnesium and 190.5 milligrams of citric oxide to 10 milliliters of water.

10. The method of claim 9, wherein the step of adding an amount of citric acid to an amount of magnesium oxide to form a second solution comprises stirring at 90 degrees Celsius.

11. The method of claim 4, wherein the step of mixing the first solution and the second solution to form a combined solution comprising repeatedly adding a portion of the second solution to the first solution over a predetermined time period until all of the second solution has been added.

12. The method of claim 11, wherein the predetermined time period is five minutes.

13. The method of claim 12, wherein the step of drying the combined solution to obtain the magnesium supplement comprises drying the combined solution in a vacuum.

14. The method of claim 12, wherein the step of drying the combined solution to obtain the magnesium supplement comprises spray drying the combined solution.
